(19) United States
(12) Patent Application Publication
(10) Pub. No.: US 2022/0008308 A1
(43) Pub. Date: Jan. 13, 2022

(54) BIS(TRIETHOXYSILYLPROPYL)AMINES COMBINED WITH POYSACCHARIDES FOR CARING FOR AND SHAPING KERATINE FIBRES

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/289,185

(22) PCT Filed: Oct. 31, 2019
(86) PCT No.: PCT/EP2019/079767

§ 371 Date: Apr. 27, 2021

(30) Foreign Application Priority Data
Oct. 31, 2018 (DE) 10 2018 127 185.9

(51) Int. Cl. A61K 8/58; A61K 8/73; A61Q 5/12

(52) U.S. Cl. CPC A61K 8/585; A61Q 5/12; A61K 8/73

(57) ABSTRACT

The present disclosure relates to an active ingredient composition for shape change and surface modification of human hair. In particular, the present disclosure relates to a cosmetic composition for treating a keratinous material comprising a) at least one organic silicon compound and b) at least one polysaccharide, the cosmetic composition being particularly suitable for providing color protection.

### CROSS-REFERENCE TO RELATED APPLICATION

This application is a U.S. National-Stage entry under 35 U.S.C. § 371 based on International Application No. PCT/EP2019/079767, filed Oct. 31, 2019, which was published under PCT Article 21(2) and which claims priority to German Application No. 10 2018 127 185.9, filed Oct. 31, 2018, which are all hereby incorporated in their entirety by reference.

### TECHNICAL FIELD

The present disclosure relates to cosmetic compositions for treating a keratinous material, the composition comprising an organic silicon compound and at least one polysaccharide, and the use of the cosmetic composition.

### BACKGROUND

The external exposure of hair to chemicals from a variety of different sources poses challenges for the development of cosmetic care products. Air and water pollution adversely affects skin and hair. The presence of other air pollutants and to UV radiation can reinforce the effect of various air pollutants.

It is known that the toxicity of gaseous pollutants of the air, such as sulfur dioxide, ozone, and nitrogen oxides, especially depends on their initiator activity for free radicals, which causes damages in living beings. Free radicals are metabolic products that also occur naturally in the body. Such cases are referred to as oxidative damage. Free radicals can also cause hair damage, evinced, for example, by a reduction in the shine as well as of the grip and/or fading of the hair color.

Furthermore, frequently changing customer requests regarding a certain finish of the hair are linked to a recurrent chemical stress on the hair. For example, hair dyes stress the hair, due to which, an intensive care may be necessary.

Organic compounds of silica from the group of silanes are described in the state of the art, which comprise at least one hydroxy group and/or hydrolysable group. Due to the presence of hydroxy groups and/or hydrolysable groups, silanes are reactive substances that hydrolyze or oligomerize or polymerize in the presence of water. When using on a keratinous material, oligomerization or polymerization of the silanes initiated by the presence of water eventually causes the formation of a film that can provide protection.

### BRIEF SUMMARY

This disclosure provides a cosmetic composition for the treatment of a keratinous material, comprising

a) at least one organic silicon compound and
b) at least one polysaccharide.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The task underlying the present disclosure includes the provision of a cosmetic product that makes it possible to shape hair well and improves the structure protection against chemical stress.

This objective is achieved by employing a cosmetic product for treating a keratinous material, comprising

a) at least one organic silicon compound and
b) at least one polysaccharide.

By a keratinous material is meant hair, the skin, the nails (such as fingernails and/or toenails). Wool, furs, and feathers also fall under the definition of keratinous material.

Preferably, a keratinous material is understood to mean human hair, human skin, and human nails, in particular fingernails and toenails. Very preferably, keratinous material is understood to mean human hair, in particular head and/or beard hair.

As a first ingredient essential to the disclosure, the cosmetic composition for treating a keratinous material contains at least one organic silicon compound. Preferred organic silicon compounds are selected from silanes with one, two or three silicon atoms, where the organic silicon compound comprises one or more hydroxyl groups and/or hydrolysable groups per molecule.

Organic silicon compounds, alternatively called organosilicon compounds, are compounds which either have a direct silicon-carbon bond (Si—C) or in which the carbon is bonded to the silicon atom via an oxygen, nitrogen, or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

The agent for treating a keratinous material contains at least one organic silicon compound preferably selected from silanes having one, two or three silicon atoms, wherein the organic silicon compound comprises one or more hydroxyl groups or hydrolysable groups per molecule.

As part of a particularly preferred embodiment, the product for the treatment of a keratinous material features at least one organic silicon compound, selected from silanes having one, two or three silicon atoms, the organic silicon compound further comprising one or more basic groups and one or more hydroxyl groups or hydrolysable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a di(C₁-C₆)alkylamino group.

The hydrolysable group(s) is (are) preferably a C₁-C₆ alkoxy group, especially an ethoxy group or a methoxy group. It is preferred when the hydrolysable group is directly bonded to the silicon atom. For example, if the hydrolysable group is an ethoxy group, the organic silicon compound preferably contains a structural unit R′R″R′″Si—O—CH₂—CH₃. The residues R′, R″ and R′″ represent the three remaining free valences of the silicon atom.

Particularly good results were obtained when the agent for treating a keratinous material contains at least one organic silicon compound of formula (I) and/or (II).

The compounds of formulae (I) and (II) are organic silicon compounds selected from silanes having one, two or three silicon atoms, the organic silicon compound comprising one or more hydroxyl groups and/or hydrolysable groups per molecule.

In another very particularly preferred embodiment, the agent for treating a keratinous material comprises at least one organic silicon compound of formula (I) and/or (II),

R₁R₂N-L-Si(OR₃)ₐ(R₄)_{b} (I),
where
R1, R2 both represent a hydrogen atom,
L represents a linear, divalent C₁-C₆-alkylene group, preferably a propylene group (—CH₂—CH₂—CH₂—) or an ethylene group (—CH₂—CH₂—),
R₃, R₄ independently represent a methyl group or an ethyl group,
a stands for the number about 3 and
b stands for the number about 0.

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c}, (II),
where
R5, R5′, R5″ independently represent a hydrogen atom or a C₁-C₆ alkyl group,
R6, R6′ and R6″ independently represent a C₁-C₆ alkyl group,
A, A′, A″, A′″ and A″″ independently represent a linear or branched double-bonded C₁-C₂₀ alkylene group,
R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

(A″″)-Si(R₆″)_{d}″(OR₅″)_{c}″ (III),
c, stands for an integer from about 1 to about 3,
d stands for the integer about 3−c,
c′ stands for an integer from about 1 to about 3,
d′ stands for the integer about 3−c′,
c″ stands for an integer from about 1 to about 3,
d″ stands for the integer about 3−c″,
e stands for about 0 or about 1,
f stands for about 0 or about 1,
g stands for about 0 or about 1,
h stands for about 0 or about 1,
provided that at least one of e, f, g, and h is different from about 0.

The substituents R₁, R₂, R₃, R₄, R₅, R₅′, R₅″, R₆, R₆′, R₆″, R₇, R₈, L, A′, A″″ and A″″ in the compounds of formula (I) and (II) are explained below as examples:

Examples of a C₁-C₆ alkyl group are the groups methyl, ethyl, propyl, isopropyl, n-butyl, s-butyl, and t-butyl, n-pentyl and n-hexyl. Propyl, ethyl, and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear divalent C₁-C₂₀ alkylene group include the methylene group (—CH₂)), the ethylene group (—CH₂—CH₂—), the propylene group (—CH₂—CH₂—CH₂—) and the butylene group (—CH₂—CH₂—CH₂—). The propylene group (—CH₂—CH₂—CH₂—) is particularly preferred. From a chain length of 3 C atoms, double-bonded alkylene groups can also be branched. Examples of branched divalent C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, divalent C₁-C₂₀ alkylene group.

Preferably -L- stands for a linear, double-bonded C₁-C₂₀ alkylene group. Further preferably -L- stands for a linear double-bonded C₁-C₆ alkylene group. Particularly preferred -L stands for a methylene group (CH₂—), an ethylene group (—CH₂—CH₂—), propylene group (—CH₂—CH₂—CH₂—) or butylene (—CH₂—CH₂—CH₂—CH₂—). L stands for a propylene group (—CH₂—CH₂—CH₂—)

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or C₁-C₆ alkyl group, and R₄ is C₁-C₆ alkyl group. R3 and R₃ independently of each other represent a methyl group or an ethyl group.

Here a stands for an integer from about 1 to about 3, and b stands for the integer about 3−a. If a stands for the number about 3, then b is equal to about 0. If a stands for the number about 2, then b is equal to about 1. If a stands for the number about 1, then b is equal to about 2.

The best protection against the negative effects of water and/or air pollution (“anti-pollution” effect) and the best care of stressed hair could be obtained if the agent for treating a keratinous material contains at least one organic silicon compound of formula (I) in which the radicals R3, R4 independently represent a methyl group or an ethyl group.

Especially well-suited organic silicon compounds of the formula (I) are:

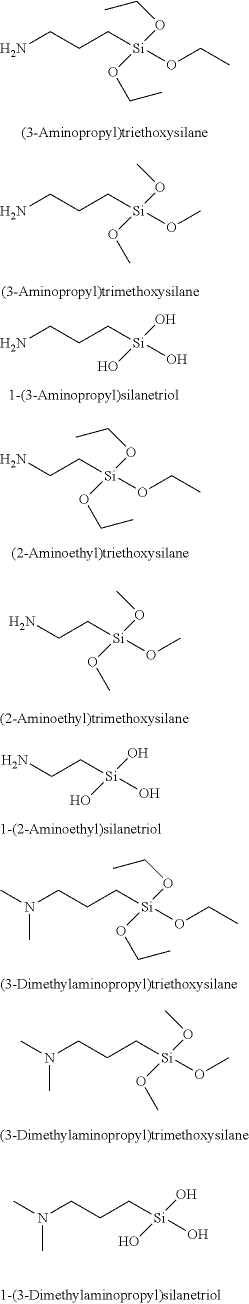

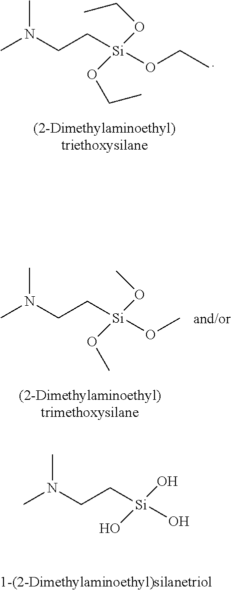

The organic silicon compound of formula (I) is commercially available. (3-aminopropyl)trimethoxysilane, for example, can be purchased from Sigma-Aldrich. (3-aminopropyl)triethoxysilane is also commercially available from Sigma-Aldrich.

In a further embodiment, the product for the treatment of a keratinous material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II).

The organosilicon compounds of formula (II) each bear at their two ends the silicon-containing groupings (R₅O)_{c}(R₆)_{d}Si— and —Si(R₆′)_{d′}(OR₅′)_{c′},

In the central part of the molecule of formula (II) there are the groups -(A)ₑ- and —[NR₇-(A′)]_{f}- and —[O-(A″)]_{g}- and —[NR₈-(A′″)]ₕ-. Here, each of the radicals e, f, g, and h can independently of one another stand for the number about 0 or about 1, with the proviso that at least one of the radicals e, f, g, and h is different from about 0. In other words, an organic silicon compound of formula (II) contains at least one grouping selected from the group including -(A)- and —[NR₇-(A′)]- and —[O-(A″)]- and —[NR₈-(A′″)]-.

In the two terminal structural units (R₅O)_{c}(R₆)_{d}Si— and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 about to about 3, and d stands for the integer about 3−c. If c stands for the number about 3, then d is equal to about 0. If c stands for the number about 2, then d is equal to about 1. If c stands for the number about 1, then d is equal to about 2.

Analogously c′ stands for a whole number from about 1 to about 3, and d′ stands for the whole number about 3−c′. If c′ stands for the number about 3, then d′ is about 0. If c′ stands for the number about 2, then d′ is about 1. If c′ stands for the number about 1, then d′ is about 2.

An extremely high anti-pollution effect of the product for the treatment of a keratinous material can be obtained if the radicals c and c′ both stand for the number about 3. In this case d and d′ both stand for the number about 0.

In a further preferred embodiment, the product for the treatment of a keratinous material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆)_{d′}(OR_{5′})_{c′} (II),

where
R5 and R5′ independently represent a methyl group or an ethyl group,
c and c′ both stand for the number about 3 and
d and d′ both stand for the number about 0.

When c and c′ both represent the number about 3 and d and d′ both represent the number about 0, the organic silicon compounds correspond to formula (IIa)

(R₅O)₃Si-(A)ₑ-[NR₇-(A′)]_{f}-[O-(A″)]_{g}-[NR₈-(A′″)]ₕ—Si(OR₅′)₃ (IIa).

The radicals e, f, g, and h can independently stand for the number 0 or 1, whereby at least one radical from e, f, g, and h is different from zero. The abbreviations e, f, g, and h thus define which of the groupings -(A)e- and -[NR7-(A′)]f- and —[O-(A″)]g- and -[NR8-(A′″)]h- are in the middle part of the organic silicon compound of formula (II).

In this context, the presence of certain groupings has proved to be particularly beneficial in terms of increasing the ‘anti-pollution’ effect. Particularly good results were obtained when at least two of the residues e, f, g, and h stand for the number about 1. Especially preferred e and f both stand for the number about 1. Furthermore, g and h both stand for the number about 0.

If e and f both stand for the number about 1 and g and h both stand for the number about 0, the organic silicon compound as contemplated herein corresponds to formula (IIb)

(R₅O)_{c}(R₆)_{d}Si-(A)-[NR₇-(A′)]-Si(R₆′)_{d′}(OR₅′)_{c′} (IIb).

The radicals A, A′, A″, A′″ and A″″ independently represent a linear or branched divalent C₁-C₂₀ alkylene group. Preferably the radicals A, A′, A″, A′″ and A″″ independently of one another represent a linear, divalent C₁-C₂₀ alkylene group. Further preferably the radicals A, A′, A″, A′″ and A″″ independently represent a linear divalent C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

If the radical f represents the number about 1, then the organic silicon compound of formula (II) contains a structural grouping —[NR₇-(A′)]-.

If the radical h represents the number about 1, then the organic silicon compound of formula (II) contains a structural grouping —[NR₈-(A′″)]-.

Herein, the radicals R₇ and R₈ independently represent a hydrogen atom, a C₁-C₆ alkyl group, a hydroxy C₁-C₆-alkyl group, a C₂-C₆ alkenyl group, an amino-C₁-C₆-alkyl group, or a grouping of the formula (III)

(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number about 1 and the radical h represents the number about 0, the organic silicon compound as contemplated herein contains the grouping [NR₇-(A′)] but not the grouping —[NR₈-(A′″)]. If the residue R7 is a grouping of formula (III), the agent for treating a keratin material contains an organic silicon compound with about 3 reactive silane groups.

In a further preferred embodiment, the product for the treatment of a keratinous material contains at least one organic silicon compound of formula (II)

(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),

where
e and f both stand for the number about 1,
g and h both stand for the number about 0,
A and A′ independently of one another represent a linear, divalent C₁-C₆ alkylene group and
R7 represents a hydrogen atom, a methyl group, a 2-hydroxyethyl group, a 2-alkenyl group, a 2-aminoethyl group or a group of formula (III).

In a further preferred embodiment, the product for the treatment of a keratinous material contains at least one organic silicon compound of formula (II), wherein
e and f both stand for the number about 1,
g and h both stand for the number about 0,
A and A′ independently of one another represent a methylene group (—CH₂—), an ethylene group (—CH₂—CH₂—) or a propylene group (—CH₂—CH₂—CH₂),
   and
R₇ represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem are

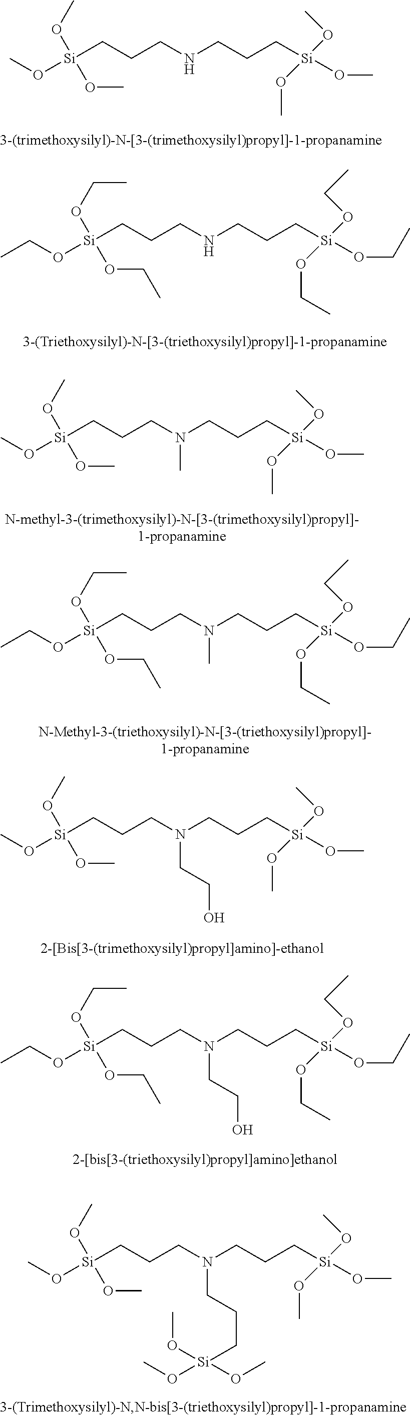

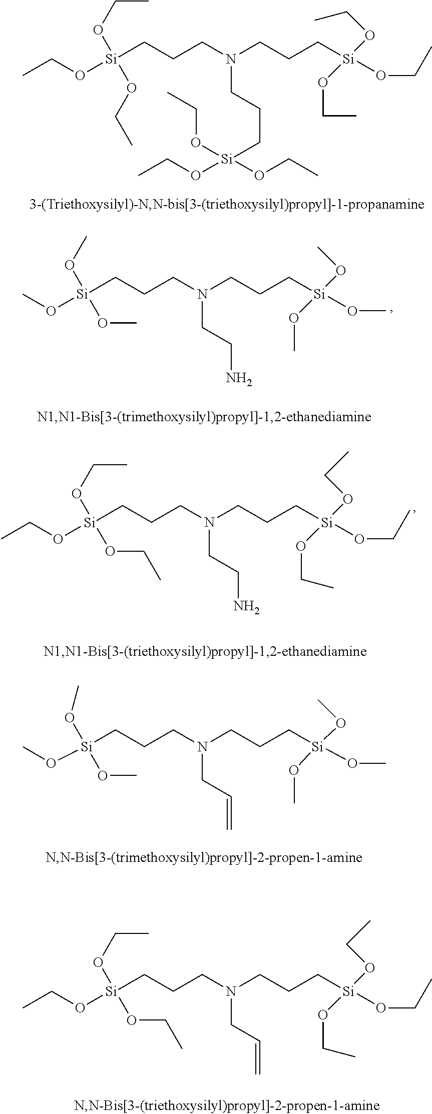

The organic silicon compounds of formula (II) are commercially available.

Bis(trimethoxysilylpropyl)amines with the CAS number 82985-35-1 can be purchased from Sigma-Aldrich.

Bis[3-(triethoxysilyl)propyl]amine, also designated as 3-(Triethoxysilyl)-N-[3-(triethoxysilyl)propyl]-1-propanamine, with the CAS number 13497-18-2 is, for example, commercially available from Sigma-Aldrich or can be commercially obtained from Evonik under the product name Dynasylan 1122.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also proved advantageous, if the product for the treatment of a keratinous material, used on the hair, contains at least one organic silicon compound of the formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₀)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer about 3−k.

In a further preferred embodiment, the product for the treatment of a keratinous material contains, in addition to the organic silicon compound(s) of formula (I), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer about 3−k.

In a similarly preferred embodiment, the product for the treatment of a keratinous material contains, in addition to the organic silicon compound(s) of formula (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer about 3−k.

In a further preferred embodiment, the product for the treatment of a keratinous material contains, in addition to the organic silicon compound(s) of formula (I) and (II), at least one more organic silicon compound of formula (IV)

R₉Si(OR₁₀)ₖ(R₁₁)ₘ (IV),

where
R₉ represents a C₁-C₁₂ alkyl group,
R₁₀ represents a hydrogen atom or a C₁-C₆ alkyl group,
R₁₁ represents a C₁-C₆ alkyl group
k is an integer from about 1 to about 3, and
m stands for the integer about 3−k.

In the organic silicon compounds of formula (IV), the radical R₉ represents a C₁-C₁₂ alkyl group. This C₁-C₁₂ alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a hydrogen atom or a C₁-C₆ alkyl group. Especially preferably, R₁₀ stands for a methyl group or an ethyl group.

In the organic silicon compounds of formula (IV), the radical R₁₁ represents a C₁-C₆ alkyl group. Particularly preferably, R₁₁ represents a methyl group or an ethyl group.

Furthermore, k stands for a whole number from about 1 to about 3, and m stands for the whole number about 3−k. If k stands for the number about 3, then m is equal to about 0. If k stands for the number about 2, then m is equal to about 1. If k stands for the number about 1, then m is equal to about 2.

An extremely high “anti-pollution” effect can be achieved if the product for the treatment of a keratinous material contains at least one organic silicon compound of the formula (IV) in which the radical k stands for the number about 3. In this case the rest m stands for the number about 0.

Organic silicon compounds of the formula (IV) which are well suited for solving the problem are

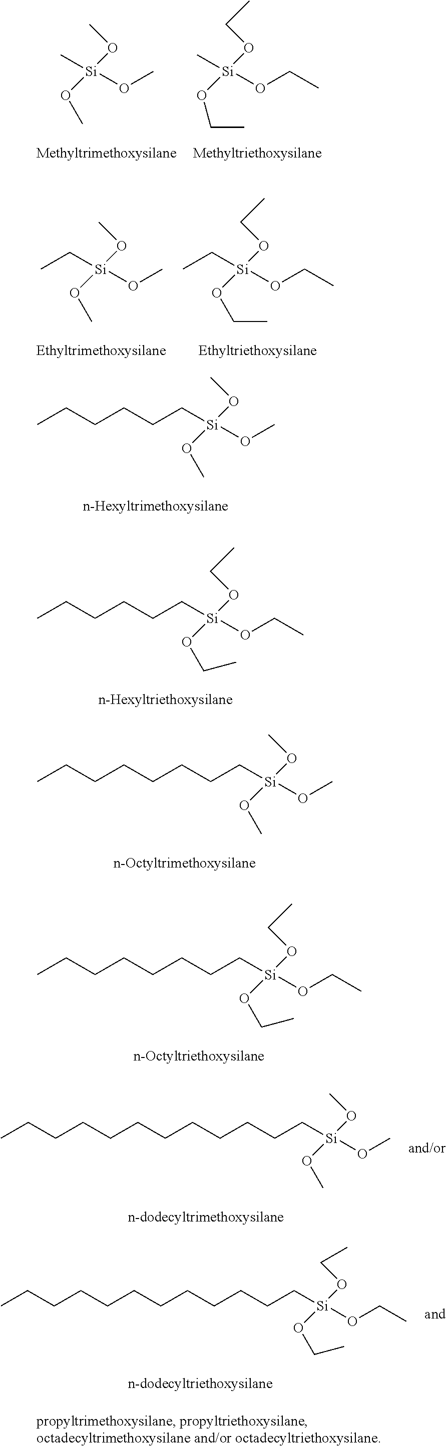

The organic silicon compounds described above are reactive compounds.

In this context, it has proved to be specifically preferable, if the product contains (3-Aminopropyl)triethoxysilane, i.e., an aminopropyl triethoxysilane (AMEO), and/or 3-(Triethoxysilyl)-N-[3-(triethoxysilyl)propyl]-1-propane amine, i.e., a Bis(trimethoxysilylpropyl)amine, as organic silicon compound.

According to preferred embodiments of the present disclosure, the organic silicon compound of formula (I), in particular the (3-aminopropyl)triethoxysilane, is present in an amount of from about 0.01 to about 10 wt. %, preferably from about 0.02 to about 8—by weight, more preferably from about 0.05 to about 6-% by weight, most preferably from about 0.1 to about 4-% by weight, based on the total weight of the cosmetic product, in the cosmetic product, and/or the organic silicon compound of the formula (II), in particular the 3-(triethoxysilyl)-N-[3-(triethoxysilyl)propyl]-1-propanamine, is present in an amount of from about 0.01 to about 10% by weight, preferably from about 0.02 to about 9—by weight, more preferably from about 0.05 to about 8-% by weight, most preferably from about 0.1 to about 7-%, by weight, based on the total weight of the cosmetic composition, in the cosmetic composition.

It was found that particularly stable and uniform films could be obtained on the keratinous material even when the agent contained two structurally different organic silicon compounds.

In a preferred embodiment, an agent is exemplified in that it comprises at least one organic silicone compound of formula (I) and at least one organic silicone compound of formula (IV).

In an explicitly very particularly preferred embodiment, an agent is exemplified in that it contains at least one organic silicone compound of formula (I) selected from the group including (3-aminopropyl)triethoxysilane and (3-aminopropyl)trimethoxysilane, and additionally containing at least one organic silicone compound of formula (IV) selected from the group including methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In another preferred embodiment, an agent is exemplified in that the agent—contains, based on the total weight of the agent-:
From about −0.5 to about 5 weight % of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
From about 3.2 to about 10 wt. % of at least one second organic silicon compound selected from the group including methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

Even the addition of small amounts of water leads to hydrolysis in organic silicon compounds with at least one hydrolysable group. The hydrolysis products and/or organic silicon compounds having at least one hydroxy group may react with each other in a condensation reaction. For this reason, both the organosilicon compounds having at least one hydrolysable group and their hydrolysis and/or condensation products may be present in the composition. When organosilicon compounds having at least one hydroxyl group are used, both the organic silicon compounds having at least one hydroxyl group and their condensation products may be present in the composition.

A condensation product is understood to be a product formed by the reaction of at least two organic silicon compounds each having at least one hydroxyl group or hydrolysable group per molecule with elimination of water and/or with elimination of an alkanol. The condensation products can be, for example, dimers, but also trimers or oligomers, with the condensation products being in equilibrium with the monomers. Depending on the amount of water used or consumed in the hydrolysis, the equilibrium shifts from monomeric organic silicon compounds to condensation product.

In the context of the present disclosure, figures in wt.-% are—unless otherwise stated—always based on the total weight of the cosmetic product.

As a second ingredient essential to the disclosure, the cosmetic composition for treating a keratinous material contains at least one polysaccharide. The at least one polysaccharide that may be included in the composition according to the present disclosure may be of plant or animal origin or a product of the metabolism of microorganisms and a degradation and modification product thereof. In the context of the present disclosure, a polysaccharide is to be understood as a polymer in which more than about two, preferably more than about 10 monosaccharides (simple sugars) are linked via a glycosidic bond. In a preferred embodiment, the monosaccharides may be used in an opened or closed ring structure.

In the course of the work leading to the present disclosure, it has been found that in order to achieve advantageous properties with regard to the shaping of the hair while at the same time protecting the structure, it is advantageous if the organic silicon compounds, in particular 3-(triethoxysilyl)-N-[3-(triethoxysilyl)propyl]-1-propanamine, i.e. i.e. a bis(trimethoxysilylpropyl)amine, and/or (3-aminopropyl)triethoxysilane, i.e. an aminopropyltriethoxysilane (AMEO), is combined with the polysaccharide.

In preferred embodiments, the polysaccharide is composed of monomers selected from the group including glyceraldehyde, erythrose, threose, ribose, Arabinose, xylose, glucose, mannose, lyxose, allose, altrose, gulose, idose, galactose, talose, dihydroxyacetone, erythrulose, ribulose, xylulose, psicose, fructose, sorbose and tagatose, The polysaccharide is further preferably selected from the group including glycogen, starch, callose, cellulose, tunicin, pectin, algin, inulin, chitin, chitosan, heparin, carrageenan, agar, gum arabic, traganth, locust bean gum, karaya gum, ghatti gum, guar gum, tara gum, gellan gum, xanthan gum, dextran, sucrose, nigeran gum and pentosans such as xylan and araban, derivatives and mixtures thereof.

Furthermore, the polysaccharide may be composed of monomers, which may be modified. According to preferred embodiments, the polysaccharide is built up from monomers that have been subjected to oxidation, reduction, sulfation, sulfonation, amidation, amination, alkylation, phosphatation, phosphonation, carboxylic acid esterification or etherification. This means that one or more monomers were first subjected to one of said treatments and then polymerized to form a polysaccharide, or that the polysaccharide was subjected to one of said treatments.

According to some embodiments, it may further be, for example, glycogen, starch (amylose and amylopectin), callose, cellulose, tunicin, pectin(s), algin, inulin, chitin, chitosan, heparin, carrageenan, agar, gum arabic, traganth, locust bean gum, karaya gum, ghatti gum, guar gum, tara gum, gellan, xanthan, dextran, sucrose, Nigeran and pentosans such as xylan and araban, whose main components include D-glucuronic acid, D-galacturonic acid, D-galacturonic acid methyl ester, D-mannuronic acid, L-guluronic acid, D- and L-galactose, 3,6-anhydro-D-galactose, L-rhamnose and D-glucoronic acid, 2-amino-2-deoxi-D-glucose and 2-amino-2-deoxi-D-galactose and their N-acetyl derivatives. Derivatives of the above, such as mixtures of the above, are also suitable. Chemically modified polysaccharides, i.e., those that have undergone any of the above treatments, include, without limitation, propyl cellulose, methyl ethyl cellulose, carboxymethyl cellulose, hydroxyethyl cellulose, methyl hydroxyethyl cellulose, ethyl hydroxyethyl cellulose, hydroxypropylcellulose, hydroxypropylmethylcellulose, carboxymethylhydroxyethylcellulose, sulfoethylcellulose, carboxymethylsulfoethylcellulose, hydroxypropylsulfoethylcellulose, hydroxyethylsulfoethylcellulose, methyl ethylhydroxyethylcellulose, methlylsulfoethylcellulose and ethylsulfoethylcellulose.

According to some embodiments, the at least one polysaccharide is preferably starch, thermally and/or mechanically treated starch, oxidatively, hydrolytically or oxidized enzymatically degraded starches, oxidized hydrolytically, or oxidized enzymatically degraded starches, and chemically modified starches. In this context, all starches are suitable in principle. According to some embodiments, these can be for example, but not restricted to, starch from corn, wheat, rice, peas, barley, rye, cassava, tapioca, sweet potatoes, or potatoes, in some embodiment, for example, native starches are preferably used. The designation native starch refers to the starch that is accessed from natural sources, such as the above-mentioned ones. A native starch is a common commercial product and is, therefore, easily available.

According to one embodiment, the at least one polysaccharide is corn, wheat, rice, pea, barley, rye, cassava, tapioca, sweet potato, or potato starch. According to one embodiment, the at least one polysaccharide is the corresponding native starch of the type. Mixtures of the are also suitable. According to one embodiment, the at least one polysaccharide is preferably corn starch, in particular native corn starch. The most preferred is the corn starch Collamyl 8412 from the company Agrabne Wien.

The combination of the organic silicon compound, for example the bis(trimethoxysilylpropyl)amine and/or the aminopropyltriethoxysilane, with the polysaccharide forms a layer on the hair. This ensures that the hair can be well shaped temporarily and a good hairstyle hold is achieved. Furthermore, the hair surface is re-hydrophobized in oxidatively damaged hair, which leads to the reduction of frizz. In addition, the structural protection against chemical action is improved.

To cope with the different requirements for the product for the treatment of a keratinous material in the form of one agent for temporary shaping of a keratinous material (=Styling material), a multitude of synthetic polymers has already been developed as solidifying compounds, which can be used in a product for the treatment of a keratinous material. It has been found that the polysaccharide used in the cosmetic composition according to claim 1 is well suited, in combination with the organic silicon compounds, to sufficiently solve the problem underlying the disclosure.

According to a preferred embodiment of the present disclosure, the amount of polysaccharide is from about 0.1 to about 25-% by weight, preferably from about 0.15 to about 20-% by weight, more preferably from about 0.5 to about 15-% by weight, most preferably from about 1 to about 10% by weight, based on the total weight of the cosmetic product, in the cosmetic product.

In the following, further ingredients of the cosmetic product are described, which may be contained in the products in addition to the previously described mandatory and optional ingredients.

It can be preferable that the product for the treatment of a keratinous material further comprise from about 0.001 to about 20 weight % of at least one quaternary compound. This holds especially for agents that lend care properties to the keratinous material.

It is preferred that the at least one quaternary compound be selected from at least one of the groups including

i) the monoalkyl quats and/or
Ii) the ester quats and/or
Iii) the quaternary imidazoline of the formula (Tkat2).

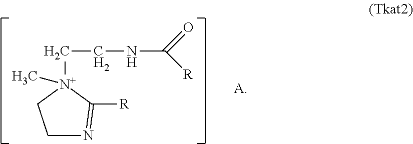

In which the radical R independently stands for a saturated or unsaturated, linear, or branched hydrocarbon radical with a chain length from about 8 to about 30 carbon atoms and A stands for a physiologically compatible anion, and/or
iv) the amidoamines and/or cationized amidoamines and/or
V) poly(methacryloyloxyethyltrimethyl ammonium compounds) and/or
vi) quaternized cellulose derivatives, especially polyquaternium 10, polyquaternium-24, polyquaternium-27, polyquaternium-67, polyquaterium-72 and/or
vii) Cationic alkyl polyglycosides and/or
viii) Cationized honey and/or
ix) Cationic guar derivatives and/or
x) Chitosan and/or
xi) quaternized polyvinyl alcohol,
xii) and mixtures thereof.

Furthermore, the cosmetic product can contain solidifying compounds, preferably selected from the group including waxes and/or other synthetic polymers. That means, waxes and/or other synthetic polymers can be added solidifying compounds as supplement. Ideally, the polysaccharides and other solidifying compounds, when used on the keratinous material, leave a polymer film that lends a strong hold to the hairstyle on the one hand, is however sufficiently flexible on the other, so that it does not break on being stressed.

For example, polycarboxylic acids can be used in the cosmetic product as film formers. These are deposited on the hair and establish a temporary deformability of the hair. The film former can be a homopolymer or copolymer derived from itaconic acid. If the film former is present exclusively in the form of polymerized itaconic acid and/or a salt of itaconic acid, the film former forms a homopolymer. An example of such a polymer is PVP/VA/Itaconic Acid Copolymer (INCI).

The other synthetic polymers can be divided in cationic, anionic, nonionic, and amphoteric solidifying polymers.

Suitable synthetic polymers comprise, for example, polymers with the following INCI designation. Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxyesters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Crosspolymer, Diphenyl Amodimethicone, Ethyl Ester of PVM/MA Copolymer, Hydrolyzed Wheat Protein/PVP Crosspolymer, Isobutylene/Ethylmaleimide/Hydroxyethylmaleimide Copolymer, Isobutylene/MA Copolymer, Isobutylmethacrylate/Bis-Hydroxypropyl Dimethicone Acrylate Copolymer, Isopropyl Ester of PVM/MA Copolymer, Lauryl Acrylate Crosspolymer, Lauryl Methacrylate/Glycol Dimethacrylate Crosspolymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Crosspolymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilylcarbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethylmethacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers such as hydroxypropyl cellulose, hydroxyethyl cellulose and methyl hydroxypropyl cellulose are also suitable.

Preferably, the solidifying compound comprises a vinylpyrrolidone-containing polymer. Particularly preferably, the solidifying compound comprises a polymer selected from the group including polyvinylpyrrolidone (PVP), vinylpyrrolidone-vinylacetate-copolymer (VP/VA copolymer), vinyl caprolactam/VP/dimethylaminoethyl methacrylate copolymer (INCI), VP/DMAPA acrylates copolymer (INCI) and mixtures thereof.

Accordingly, it is particularly preferred that the firming compound comprises a synthetic polymer selected from the group including polyvinylpyrrolidone (PVP), vinylpyrrolidone-vinyl acetate copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI), and mixtures thereof.

Furthermore, the cosmetic product can contain an Octylacrylamide/Acrylates/Butylaminoethyl Methacrylate Copolymer (INCI) as a solidifying polymer. This copolymer is sold by Akzo Nobel under the names “Amphomer®”, “Amphomer® HC”, “Amphomer® 4961”, “Amphomer® EDGE” and “Amphomer® LV 71” in various modifications.

The product for the treatment of a keratinous material can comprise especially an agent for temporarily shaping a keratinous material, an agent for cleaning a keratinous material, an agent for care of a keratinous material, an agent for care and/or an agent for care and cleaning of a keratinous material.

The cosmetic composition can, additionally or alternatively to a synthetic polymer, contain at least one natural or synthetic wax, which has a melting point above about 37° C., as solidifying compound.

As natural or synthetic waxes, solid paraffins or isoparaffins, vegetable waxes such candelila wax, carnauba wax, esparto grass wax, Japan wax, cork wax, sugar cane wax, ouricury wax, montan wax, sunflower wax, fruit waxes and animal waxes such as beeswaxes and other insect waxes, whale wax, shellac wax, wool wax and brushing grease, furthermore mineral waxes such as Ceresin and Ozokerite or the petrochemical waxes such as petrolatum, paraffin wax, microwaxes from polyethylene or polypropylene and polyethylene glycol waxes can be used. It can be advantageous to use hydrated or hardened waxes. Furthermore, chemically modified waxes, especially resin waxes, for example montan ester waxes, sasol waxes and hydrated jojoba waxes can also be used.

Furthermore, the triglycerides of saturated and, in some cases, hydroxylized C 16-30 fatty acids, such as hardened triglyceride fats (hydrated palm oil, hydrated coconut oil, hydrated castor oil), glyceryl tribehenate or glyceryl tri-12 hydroxy stearate, are suitable.

The wax components can also be selected from the group of esters of saturated, unbranched alkanecarboxylic acids having a chain length of about 22 to about 44 carbon atoms and saturated, unbranched alcohols having a chain length of about 22 to about 44 carbon atoms, provided that the wax component or the totality of wax components are solid at room temperature. Silicone waxes, for example stearyltrimethylsilane/stearyl alcohol, may also be beneficial.

Natural, chemically modified, and synthetic waxes can be used alone or in combination. Thus, several waxes can also be used. Moreover, a series of wax mixes, possibly in mixture with other additives, is also commercially available. Examples of usable mixtures are the ones available under the names “Special wax 7686 OE’ (a mixture of cetyl palmitate, bees wax, micro crystalline wax and polyethylene with a melting range of about 73-75° C., manufacturer: Kahl & Co), Plywax® GP 200 (a mixture of stearyl alcohol and polyethylene glycol stearate with melting point of about 47-51° C.; manufacturer: Croda) and “Weichceresin® FL 400” (a vaseline/vaseline oil/wax mixture with a melting point of about 50-54° C.; manufacturer: Parafluid Mineralölgesellschaft).

The wax selected from Carnauba wax (INCI: Copernicia Cerifera Cera) Bienenwachs (INCI: Beeswax), petrolatum (INCI), microcrystalline wax and especially mixtures thereof.

Preferred blends include the combination of carnauba wax (INCI: Copernicia Cerifera Cera), petrolatum and microcrystalline wax or the combination of beeswax (INCI: Beeswax) and petrolatum.

The wax or wax components should be solid at about 25° C. and should melt in the range >37° C.

The product for the treatment of a keratinous material contains a solidifying compound preferably in a total quantity of about 0.5 to about 50 weight %, preferably from about 1 to about 40 weight %, more preferred from about 1.5 to about 30 weight %, still more preferred from about 2 to about 25 weight %, based on the total weight of the cosmetic composition.

Other suitable ingredients comprise nonionic polymers, anionic polymers, (other) cationic polymers, waxes, protein hydrolysates, amino acids, oligopeptides, vitamins, pro-vitamins, vitamin precursors, betaines, biochinones, purine (derivatives), care substances, plant extracts, silicones, ester oils, UV light protection filter, thickeners, electrolytes, pH adjusters, swelling agents, dyes, anti-dandruff agents, complexing agents, opacifiers, pearlizing agents, pigments, stabilizers, propellants, anti-oxidants, perfume oils and/or preservatives.

In preferred embodiments 1 to 138, the preferred organic silicon compounds of the following table are combined with a polysaccharide composed of the monomers of the following table. The following combinations are present in cosmetic compositions in preferred embodiments of the present disclosure with other ingredients described above:

| | | |
|---|---|---|
| 1 | 3-(Triethoxysilyl)-N-[3- | Glycerinaldehyde |
| | (triethoxysilyl)propyl]-1-propanamine | |
| 2 | 3-(Triethoxysilyl)-N-[3- | Erythrose |
| | (triethoxysilyl)propyl]-1-propanamine | |
| 3 | 3-(Triethoxysilyl)-N-[3- | Threose |
| | (triethoxysilyl)propyl]-1-propanamine | |
| 4 | 3-(Triethoxysilyl)-N-[3- | Ribose |
| | (triethoxysilyl)propyl]-1-propanamine | |
| 5 | 3-(Triethoxysilyl)-N-[3- | Arabinose |
| | (triethoxysilyl)propyl]-1-propanamine | |
| 6 | 3-(Triethoxysilyl)-N-[3- | Xylose |
| | (triethoxysilyl)propyl]-1-propanamine | |
| 7 | 3-(Triethoxysilyl)-N-[3- | Glucose |
| | (triethoxysilyl)propyl]-1-propanamine | |
| 8 | 3-(Triethoxysilyl)-N-[3- | Mannose |
| | (triethoxysilyl)propyl]-1-propanamine | |
| 9 | 3-(Triethoxysilyl)-N-[3- | Lyxose |
| | (triethoxysilyl)propyl]-1-propanamine | |
| 10 | 3-(Triethoxysilyl)-N-[3- | Allose |
| | (triethoxysilyl)propyl]-1-propanamine | |
| 11 | 3-(Triethoxysilyl)-N-[3- | Altrose |
| | (triethoxysilyl)propyl]-1-propanamine | |
| 12 | 3-(Triethoxysilyl)-N-[3- | Gulose |
| | (triethoxysilyl)propyl]-1-propanamine | |
| 13 | 3-(Triethoxysilyl)-N-[3- | Idose |
| | (triethoxysilyl)propyl]-1-propanamine | |
| 14 | 3-(Triethoxysilyl)-N-[3- | Galactose |
| | (triethoxysilyl)propyl]-1-propanamine | |
| 15 | 3-(Triethoxysilyl)-N-[3- | Talose |
| | (triethoxysilyl)propyl]-1-propanamine | |
| 16 | 3-(Triethoxysilyl)-N-[3- | Dihydroxyacetone |
| | (triethoxysilyl)propyl]-1-propanamine | |
| 17 | 3-(Triethoxysilyl)-N-[3- | Erythrulose |
| | (triethoxysilyl)propyl]-1-propanamine | |
| 18 | 3-(Triethoxysilyl)-N-[3- | Ribulose |
| | (triethoxysilyl)propyl]-1-propanamine | |
| 19 | 3-(Triethoxysilyl)-N-[3- | Xylulose |
| | (triethoxysilyl)propyl]-1-propanamine | |
| 20 | 3-(Triethoxysilyl)-N-[3- | Psicose |
| | (triethoxysilyl)propyl]-1-propanamine | |
| 21 | 3-(Triethoxysilyl)-N-[3- | Fructose |
| | (triethoxysilyl)propyl]-1-propanamine | |
| 22 | 3-(Triethoxysilyl)-N-[3- | Sorbose |
| | (triethoxysilyl)propyl]-1-propanamine | |
| 23 | 3-(Triethoxysilyl)-N-[3- | Tagatose |
| | (triethoxysilyl)propyl]-1-propanamine | |
| 24 | (3-Aminopropyl)triethoxysilane | Glycerinaldehyde |
| 25 | (3-Aminopropyl)triethoxysilane | Erythrose |
| 26 | (3-Aminopropyl)triethoxysilane | Threose |
| 27 | (3-Aminopropyl)triethoxysilane | Ribose |
| 28 | (3-Aminopropyl)triethoxysilane | Arabinose |
| 29 | (3-Aminopropyl)triethoxysilane | Xylose |
| 30 | (3-Aminopropyl)triethoxysilane | Glucose |
| 31 | (3-Aminopropyl)triethoxysilane | Mannose |
| 32 | (3-Aminopropyl)triethoxysilane | Lyxose |
| 33 | (3-Aminopropyl)triethoxysilane | Allose |
| 34 | (3-Aminopropyl)triethoxysilane | Altrose |
| 35 | (3-Aminopropyl)triethoxysilane | Gulose |
| 36 | (3-Aminopropyl)triethoxysilane | Idose |
| 37 | (3-Aminopropyl)triethoxysilane | Galactose |
| 38 | (3-Aminopropyl)triethoxysilane | Talose |
| 39 | (3-Aminopropyl)triethoxysilane | Dihydroxyacetone |
| 40 | (3-Aminopropyl)triethoxysilane | Erythrulose |
| 41 | (3-Aminopropyl)triethoxysilane | Ribulose |
| 42 | (3-Aminopropyl)triethoxysilane | Xylulose |
| 43 | (3-Aminopropyl)triethoxysilane | Psicose |
| 44 | (3-Aminopropyl)triethoxysilane | Fructose |
| 45 | (3-Aminopropyl)triethoxysilane | Sorbose |
| 46 | (3-Aminopropyl)triethoxysilane | Tagatose |
| 47 | (3-Aminopropyl)triethoxysilane + | Glycerinaldehyde |
| | Methyltrimethoxysilane | |
| 48 | (3-Aminopropyl)triethoxysilane + | Glycerinaldehyde |
| | methyltriethoxysilane | |
| 49 | (3-Aminopropyl)triethoxysilane + | Glycerinaldehyde |
| | ethyltrimethoxysilane | |
| 50 | (3-Aminopropyl)triethoxysilane + | Glycerinaldehyde |
| | ethyltriethoxysilane | |
| 51 | (3-Aminopropyl)triethoxysilane + | Erythrose |
| | Methyltrimethoxysilane | |
| 52 | (3-Aminopropyl)triethoxysilane + | Erythrose |
| | methyltriethoxysilane | |
| 53 | (3-Aminopropyl)triethoxysilane + | Erythrose |
| | ethyltrimethoxysilane | |
| 54 | (3-Aminopropyl)triethoxysilane + | Erythrose |
| | ethyltriethoxysilane | |
| 55 | (3-Aminopropyl)triethoxysilane + | Threose |
| | Methyltrimethoxysilane | |
| 56 | (3-Aminopropyl)triethoxysilane + | Threose |
| | methyltriethoxysilane | |
| 57 | (3-Aminopropyl)triethoxysilane + | Threose |
| | ethyltrimethoxysilane | |
| 58 | (3-Aminopropyl)triethoxysilane + | Threose |
| | ethyltriethoxysilane | |
| 59 | (3-Aminopropyl)triethoxysilane + | Ribose |
| | Methyltrimethoxysilane | |
| 60 | (3-Aminopropyl)triethoxysilane + | Ribose |
| | methyltriethoxysilane | |
| 61 | (3-Aminopropyl)triethoxysilane + | Ribose |
| | ethyltrimethoxysilane | |
| 62 | (3-Aminopropyl)triethoxysilane + | Ribose |
| | ethyltriethoxysilane | |
| 63 | (3-Aminopropyl)triethoxysilane + | Arabinose |
| | Methyltrimethoxysilane | |
| 64 | (3-Aminopropyl)triethoxysilane + | Arabinose |
| | methyltriethoxysilane | |
| 65 | (3-Aminopropyl)triethoxysilane + | Arabinose |
| | ethyltrimethoxysilane | |
| 66 | (3-Aminopropyl)triethoxysilane + | Arabinose |
| | ethyltriethoxysilane | |
| 67 | (3-Aminopropyl)triethoxysilane + | Xylose |
| | Methyltrimethoxysilane | |
| 68 | (3-Aminopropyl)triethoxysilane + | Xylose |
| | methyltriethoxysilane | |
| 69 | (3-Aminopropyl)triethoxysilane + | Xylose |
| | ethyltrimethoxysilane | |
| 70 | (3-Aminopropyl)triethoxysilane + | Xylose |
| | ethyltriethoxysilane | |
| 71 | (3-Aminopropyl)triethoxysilane + | Glucose |
| | Methyltrimethoxysilane | |
| 72 | (3-Aminopropyl)triethoxysilane + | Glucose |
| | methyltriethoxysilane | |
| 73 | (3-Aminopropyl)triethoxysilane + | Glucose |
| | ethyltrimethoxysilane | |
| 74 | (3-Aminopropyl)triethoxysilane + | Glucose |
| | ethyltriethoxysilane | |
| 75 | (3-Aminopropyl)triethoxysilane + | Mannose |
| | Methyltrimethoxysilane | |
| 76 | (3-Aminopropyl)triethoxysilane + | Mannose |
| | methyltriethoxysilane | |
| 77 | (3-Aminopropyl)triethoxysilane + | Mannose |
| | ethyltrimethoxysilane | |
| 78 | (3-Aminopropyl)triethoxysilane + | Mannose |
| | ethyltriethoxysilane | |
| 79 | (3-Aminopropyl)triethoxysilane + | Lyxose |
| | Methyltrimethoxysilane | |
| 80 | (3-Aminopropyl)triethoxysilane + | Lyxose |
| | methyltriethoxysilane | |
| 81 | (3-Aminopropyl)triethoxysilane + | Lyxose |
| | ethyltrimethoxysilane | |
| 82 | (3-Aminopropyl)triethoxysilane + | Lyxose |
| | ethyltriethoxysilane | |
| 83 | (3-Aminopropyl)triethoxysilane + | Allose |
| | Methyltrimethoxysilane | |
| 84 | (3-Aminopropyl)triethoxysilane + | Allose |
| | methyltriethoxysilane | |
| 85 | (3-Aminopropyl)triethoxysilane + | Allose |
| | ethyltrimethoxysilane | |
| 86 | (3-Aminopropyl)triethoxysilane + | Allose |
| | ethyltriethoxysilane | |
| 87 | (3-Aminopropyl)triethoxysilane + | Altrose |
| | Methyltrimethoxysilane | |
| 88 | (3-Aminopropyl)triethoxysilane + | Altrose |
| | methyltriethoxysilane | |
| 89 | (3-Aminopropyl)triethoxysilane + | Altrose |
| | ethyltrimethoxysilane | |
| 90 | (3-Aminopropyl)triethoxysilane + | Altrose |
| | ethyltriethoxysilane | |
| 91 | (3-Aminopropyl)triethoxysilane + | Gulose |
| | Methyltrimethoxysilane | |
| 92 | (3-Aminopropyl)triethoxysilane + | Gulose |
| | methyltriethoxysilane | |
| 93 | (3-Aminopropyl)triethoxysilane + | Gulose |
| | ethyltrimethoxysilane | |
| 94 | (3-Aminopropyl)triethoxysilane + | Gulose |
| | ethyltriethoxysilane | |
| 95 | (3-Aminopropyl)triethoxysilane + | Idose |
| | Methyltrimethoxysilane | |
| 96 | (3-Aminopropyl)triethoxysilane + | Idose |
| | methyltriethoxysilane | |
| 97 | (3-Aminopropyl)triethoxysilane + | Idose |
| | ethyltrimethoxysilane | |
| 98 | (3-Aminopropyl)triethoxysilane + | Idose |
| | ethyltriethoxysilane | |
| 99 | (3-Aminopropyl)triethoxysilane + | Galactose |
| | Methyltrimethoxysilane | |
| 100 | (3-Aminopropyl)triethoxysilane + | Galactose |
| | methyltriethoxysilane | |
| 101 | (3-Aminopropyl)triethoxysilane + | Galactose |
| | ethyltrimethoxysilane | |
| 102 | (3-Aminopropyl)triethoxysilane + | Galactose |
| | ethyltriethoxysilane | |
| 103 | (3-Aminopropyl)triethoxysilane + | Talose |
| | Methyltrimethoxysilane | |
| 104 | (3-Aminopropyl)triethoxysilane + | Talose |
| | methyltriethoxysilane | |
| 105 | (3-Aminopropyl)triethoxysilane + | Talose |
| | ethyltrimethoxysilane | |
| 106 | (3-Aminopropyl)triethoxysilane + | Talose |
| | ethyltriethoxysilane | |
| 107 | (3-Aminopropyl)triethoxysilane + | Dihydroxyacetone |
| | Methyltrimethoxysilane | |
| 108 | (3-Aminopropyl)triethoxysilane + | Dihydroxyacetone |
| | methyltriethoxysilane | |
| 109 | (3-Aminopropyl)triethoxysilane + | Dihydroxyacetone |
| | ethyltrimethoxysilane | |
| 110 | (3-Aminopropyl)triethoxysilane + | Dihydroxyacetone |
| | ethyltriethoxysilane | |
| 111 | (3-Aminopropyl)triethoxysilane + | Erythrulose |
| | Methyltrimethoxysilane | |
| 112 | (3-Aminopropyl)triethoxysilane + | Erythrulose |
| | methyltriethoxysilane | |
| 113 | (3-Aminopropyl)triethoxysilane + | Erythrulose |
| | ethyltrimethoxysilane | |
| 114 | (3-Aminopropyl)triethoxysilane + | Erythrulose |
| | ethyltriethoxysilane | |
| 115 | (3-Aminopropyl)triethoxysilane + | Ribulose |
| | Methyltrimethoxysilane | |
| 116 | (3-Aminopropyl)triethoxysilane + | Ribulose |
| | methyltriethoxysilane | |
| 117 | (3-Aminopropyl)triethoxysilane + | Ribulose |
| | ethyltrimethoxysilane | |
| 118 | (3-Aminopropyl)triethoxysilane + | Ribulose |
| | ethyltriethoxysilane | |
| 119 | (3-Aminopropyl)triethoxysilane + | Xylulose |
| | Methyltrimethoxysilane | |
| 120 | (3-Aminopropyl)triethoxysilane + | Xylulose |
| | methyltriethoxysilane | |
| 121 | (3-Aminopropyl)triethoxysilane + | Xylulose |
| | ethyltrimethoxysilane | |
| 122 | (3-Aminopropyl)triethoxysilane + | Xylulose |
| | ethyltriethoxysilane | |
| 123 | (3-Aminopropyl)triethoxysilane + | Psicose |
| | Methyltrimethoxysilane | |
| 124 | (3-Aminopropyl)triethoxysilane + | Psicose |
| | methyltriethoxysilane | |
| 125 | (3-Aminopropyl)triethoxysilane + | Psicose |
| | ethyltrimethoxysilane | |
| 126 | (3-Aminopropyl)triethoxysilane + | Psicose |
| | ethyltriethoxysilane | |
| 127 | (3-Aminopropyl)triethoxysilane + | Fructose |
| | Methyltrimethoxysilane | |
| 128 | (3-Aminopropyl)triethoxysilane + | Fructose |
| | methyltriethoxysilane | |
| 129 | (3-Aminopropyl)triethoxysilane + | Fructose |
| | ethyltrimethoxysilane | |
| 130 | (3-Aminopropyl)triethoxysilane + | Fructose |
| | ethyltriethoxysilane | |
| 131 | (3-Aminopropyl)triethoxysilane + | Sorbose |
| | Methyltrimethoxysilane | |
| 132 | (3-Aminopropyl)triethoxysilane + | Sorbose |
| | methyltriethoxysilane | |
| 133 | (3-Aminopropyl)triethoxysilane + | Sorbose |
| | ethyltrimethoxysilane | |
| 134 | (3-Aminopropyl)triethoxysilane + | Sorbose |
| | ethyltriethoxysilane | |
| 135 | (3-Aminopropyl)triethoxysilane + | Tagatose |
| | Methyltrimethoxysilane | |
| 136 | (3-Aminopropyl)triethoxysilane + | Tagatose |
| | methyltriethoxysilane | |
| 137 | (3-Aminopropyl)triethoxysilane + | Tagatose |
| | ethyltrimethoxysilane | |
| 138 | (3-Aminopropyl)triethoxysilane + | Tagatose |
| | ethyltriethoxysilane | |

The active ingredient combination of at least one organic silicon compound and the polysaccharide may already be present in the agent for treating a keratinous material. In this embodiment, the product for the treatment of a keratinous material is already sold in ready-to-use form. To provide a formulation that is as stable as possible during storage, the product itself is preferably packaged with low or no water.

Alternatively, the at least one organic silicon compound is added to a base comprising all ingredients of the product for the treatment of a keratinous material except for the at least one organic silicon compound, maximum about 12 hours, preferably maximum about 6 hours, more preferably maximum about 3 hours, even more preferably maximum about 1 hour before using the product for the treatment of a keratinous material.

Furthermore, alternatively, the organic silicon compound and the polysaccharide are added to a cosmetic product only shortly before use, i.e., about 1 minute to about 12 hours, preferably from about 2 minutes to about 6 hours, particularly preferably from about 1 minute to about 3 hours, especially preferably from about 1 minute to about 1 hour.

In another alternative, the organic silicon compound is added to an aqueous solution, which is applied to the hair, and in the second step, an aqueous solution or cosmetic agent containing the polysaccharide is applied to the hair.

The user can for example stir or shake an agent (α), which contains the organic silicon compound(s), first with an agent (β), which comprises the remaining ingredients of the product for the treatment of a keratinous material. The user can now apply this mixture of (α) and (β) to the keratinous materials—either directly after their production or after a short reaction time of about 1 minute to about 20 minutes. The agent (β) can contain water, especially water in a quantity >about 30 weight %, based on the total weight of the product for the treatment of keratinous materials.

Another object of the present application is the use of a cosmetic product, as disclosed, for the treatment of a keratinous material

for the washout protection of colored keratinous material,
for shaping of the surface of keratinous material and/or
for care of keratinous material.

Regarding other preferred embodiments of the use, what was said about the cosmetic product is applicable mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic composition for the treatment of a keratinous material, comprising
a) at least one organic silicon compound and
b) at least one polysaccharide.

2. A cosmetic composition for the treatment of a keratinous material as claimed in claim 1,
wherein
the at least one organic silicon compound comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, divalent C₁-C₆-alkylene group,
R₃, R₄ independently represent a methyl group or an ethyl group,
a is the number about 3 and
b is the number about 0 and
wherein in the organic silicon compound of formula (II)
(R₅O)_{c}(R₆)_{d}Si-(A)ₑ-[NR₇-(A′)]_{f}-[O-(A″)]_{g}-[NR₈-(A′″)]ₕ-Si(R₆′)_{d′}(OR₅′)_{c′} (II),
R₅, R_{5′}, R_{5″}, R₆, R_{6′} and R_{6″} independently represent a C₁-C₆ alkyl group,
A, A′, A″, A′″ and A″″ independently represent a linear or branched, double-bonded C₁-C₂₀-alkylene group,
R₇ and R₈ independently represent a hydrogen atom, a C₁-C₆-alkyl group, a hydroxy-C₁-C₆-alkyl group, a C₂-C₆-alkenyl group, an amino-C₁-C₆-alkyl group, or a grouping of the formula (III)
(A″″)-Si(R₆″)_{d″}(OR₅″)_{c″} (III),
c″ is an integer from about 1 to about 3,
d is the integer about 3−c,
c′ is an integer from about 1 to about 3,
d′ is the integer about 3−c′,
c″ is an integer from about 1 to about 3,
d″ is the integer about 3−c″,
e is about 0 or about 1,
f is about 0 or about 1,
g is about 0 or about 1,
h is about 0 or about 1,
provided that at least one of e, f, g, and h is different from about 0.

3. A cosmetic composition for the treatment of a keratinous material as claimed in claim 1,
wherein
the product for the treatment of a keratinous material comprises at least one organic silicon compound of the formula (I), which is chosen from
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and
(2-Dimethylaminoethyl)triethoxysilane,
Or wherein
the product for the treatment of a keratinous material comprises at least one organic silicon compound of the formula (II), which is chosen from
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-bis[3-(trimethoxysilyl)propyl]-1,2-ethanediamine,
N1,N1-bis[3-(triethoxysilyl)propyl]-1,2-ethanediamine,
N,N-bis[3-(trimethoxysilyl) propyl]-2-propene-1-amine and
N,N-bis[3-(triethoxysilyl)propyl]-2-propen-1-amine.

4. A cosmetic composition for the treatment of a keratinous material as claimed in claim 1, wherein the organic silicon compound of the formula (I) is included in a quantity of from about 0.01 to about 10 weight %, based on the total weight of the cosmetic composition, and/or wherein the organic silicon compound of the formula (II), is included in a quantity of from about 0.01 to about 10-% by weight based on the total weight of the cosmetic composition,
and/or wherein the organic silicon compound of formula (I) is 3-(Triethoxysilyl)-N-[3-(triethoxysilyl) propyl]-1-propane amine and/or the organic silicon compounds of formula (II) (3-Aminopropyl)triethoxysilane.

5. A cosmetic composition for treating a keratinous material according to claim 1, wherein the agent for treating a keratinous material comprises at least one organic silicon compound of formula (IV),
R₉Si(OR₁₀)k(R₁₁)ₘ (IV),
which is chosen from
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane
Octadecyltrimethoxysilane and
Octadecyltriethoxysilane.

6. A cosmetic composition for treating a keratinous material according to claim 1, wherein the polysaccharide is present in the cosmetic composition in an amount of from about 0.1 to about 25-% by weight based on the total weight of the cosmetic composition.

7. A cosmetic composition for treating a keratinous material according to claim 1, wherein the polysaccharide is composed of monomers chosen from Glycerinaldehyde, Erythrose, Threose, Ribose, Arabinose, Xylose, Glucose, Mannose, Lyxose, Allose, Altrose, Gulose, Idose, Galactose, Talose, Dihydroxyacetone, Erythrulose, Ribulose, Xylulose, Psicose, Fructose, Sorbose, Tagatose and mixtures thereof.

8. A cosmetic composition for the treatment of a keratinous material according to claim 1, wherein the polysaccharide is composed of monomers which have been subjected to oxidation, reduction, sulfation, sulfonation, amidation, amination, alkylation, phosphatation, phosphonation, carboxylic acid esterification or etherification.

9. A cosmetic composition for treating a keratinous material according to claim 1, wherein the cosmetic composition for treating a keratinous material comprises at least two organic silicon compounds which are structurally different from one another.

10. A cosmetic composition for treating a keratinous material according to claim 1, wherein the composition for treating a keratinous material comprises—based on the total weight of the composition for treating a keratinous material:
from about 0.5 to about 3 weight % of at least one first organic silicon compound chosen from (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
from about 3.2 to about 7 wt. % of at least one second organic silicon compound chosen from methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilanen, octadecyltrimethoxysilane and octadecyltriethoxysilane.

11. (canceled)

12. The cosmetic composition of claim 1 wherein the organic silicon compound of the formula (I) is included in a quantity of from about 0.02 to about 8% by weight, based on the total weight of the cosmetic composition.

13. The cosmetic composition of claim 1 wherein the organic silicon compound of the formula (I) is included in a quantity of from about 0.05 to 6 about % by weight, based on the total weight of the cosmetic composition.

14. The cosmetic composition of claim 1 wherein the organic silicon compound of the formula (I) is included in a quantity of from about 0.1 to about 4% by weight, based on the total weight of the cosmetic composition.

15. The cosmetic composition of claim 1 wherein the organic silicon compound of the formula (II) is included in a quantity of from about 0.02 to about 9% by weight, based on the total weight of the cosmetic composition.

16. The cosmetic composition of claim 1 wherein the organic silicon compound of the formula (II) is included in a quantity of from about 0.05 to about 8% by weight, based on the total weight of the cosmetic composition.

17. The cosmetic composition of claim 1 wherein the organic silicon compound of the formula (II) is included in a quantity of from about 0.1 to about 7% by weight, based on the total weight of the cosmetic composition.

18. The cosmetic composition of claim 1 wherein the organic silicon compound of the formula (II) is included in a quantity of from about 0.1 to about 6% by weight, based on the total weight of the cosmetic composition.

19. The cosmetic composition of claim 1 wherein the polysaccharide is present in an amount of from about 0.15 to 20% by weight, based on the total weight of the cosmetic composition.

20. The cosmetic composition of claim 1 wherein the polysaccharide is present in an amount of from about 0.5 to about 15% by weight, based on the total weight of the cosmetic composition.

21. The cosmetic composition of claim 1 wherein the polysaccharide is present in an amount of from about 1 to about 10% by weight, based on the total weight of the cosmetic composition.
